(19) United States
(12) Patent Application Publication
(10) Pub. No.: US 2026/0341523 A1
(43) Pub. Date: Oct. 29, 2026

(54) BIOMECHANICAL INJURY ANALYZER

(71) Applicant: Texas A&M University System, College Station, TX (US)

(72) Inventors: Srihari Menon, College Station, TX (US); Nancy Currie-Gregg, College Station, TX (US)

(21) Appl. No.: 19/187,405

(22) Filed: Apr. 23, 2025

(51) Int. Cl. G16H 50/30; G16H 50/50

(52) U.S. Cl. CPC G16H 50/30; G16H 50/50

(57) ABSTRACT

The disclosure provides a biomechanical injury analyzer that automatically analyzes simulation data generated from simulated impact events to determine risk assessment for an anatomical complex. In one example, the biomechanical injury analyzer comprises (1) a screen and (2) one or more processors to perform operations that include: calculating injury metrics and maximum forces of an anatomical complex using simulation data, wherein the simulation data is from a simulation of an impact event on a finite element human body model (FE-HBM), calculating associated site injury probabilities for each site of the anatomical complex using the injury metrics or maximum forces associated with the site, calculating a total injury probability of the anatomical complex based on the individual injury probabilities of the sites, displaying on the screen an injury heat map of the anatomical complex, and displaying on the screen the total injury probability of the anatomical complex.

### TECHNICAL FIELD

This application is directed, in general, to injury risk assessment and, more specifically, to assessing injury risks of systems in which a human can experience a dynamic load.

### BACKGROUND

Humans can experience hazardous loads during impact events, such as vehicle accidents, explosions, seat ejections from aircraft, spaceflights, etc. Understanding how humans can be injured when experiencing dynamic loads during these events is helpful when designing the systems involved, such as the vehicles, ejection seats, aircraft, and spaceships. Understanding injury possibilities from the dynamic loads can also be beneficial for engineering mitigation, establishing legal regulations, and establishing technical requirements.

One way to assess injury risks to humans due to dynamic loads is through computational analysis. As such, virtual human surrogates, referred to as finite element human body models (FE-HBMs), were developed to use for impact studies. An example of a FE-HBM that has been developed by the Global Human Body Model Consortium (GHBMC) is known as a GHBMC model.

### SUMMARY

In one aspect, the disclosure provides a biomechanical injury analyzer. In one example, the biomechanical injury analyzer comprises (1) a screen and (2) one or more processors to perform operations that include: (2A) calculating injury metrics and maximum forces of an anatomical complex using simulation data, wherein the simulation data is from a simulation of an impact event on a finite element human body model (FE-HBM), (2B) calculating associated site injury probabilities for each site of the anatomical complex using the injury metrics or maximum forces associated with the site, (2C) calculating a total injury probability of the anatomical complex based on the individual injury probabilities of the sites, (2D) displaying on the screen an injury heat map of the anatomical complex, and (2E) displaying on the screen the total injury probability of the anatomical complex.

In another aspect, the disclosure provides an injury assessment system. In one example, the injury assessment system includes: (1) a virtual solver configured to simulate impact events on a human body and provide simulation data that indicates how the human body reacts to the impact events, and (2) a biomechanical injury analyzer configured to automatically generate a total injury probability of the anatomical complex using the simulation data.

In still another aspect, the disclosure provides a method of providing a visual representation of injury probabilities of an anatomical complex. In one example the method includes: (1) calculating injury metrics and maximum forces of an anatomical complex using simulation data, wherein the simulation data is from a simulation of an impact event on a finite element human body model (FE-HBM), (2) calculating injury probabilities for each site of the anatomical complex based on the injury metrics and maximum forces, (3) generating a three dimensional, interactive heat map using the injury probabilities for each of the sites, and (4) displaying the injury heat map of the anatomical complex of a screen of a computing device.

### BRIEF DESCRIPTION

Reference is now made to the following descriptions taken in conjunction with the accompanying drawings, in which:

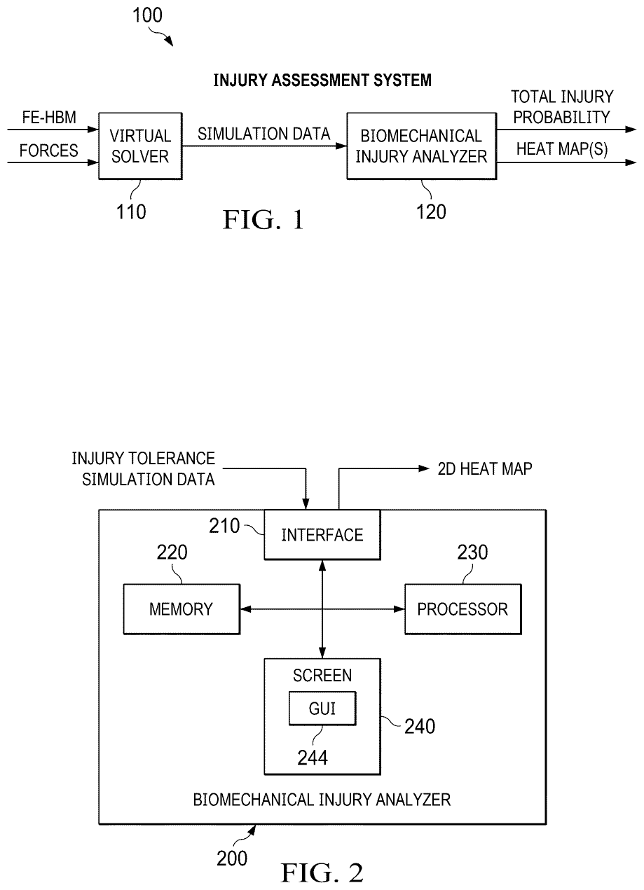

FIG. 1 illustrates a block diagram of an example of an injury assessment system constructed according to the principles of the disclosure;

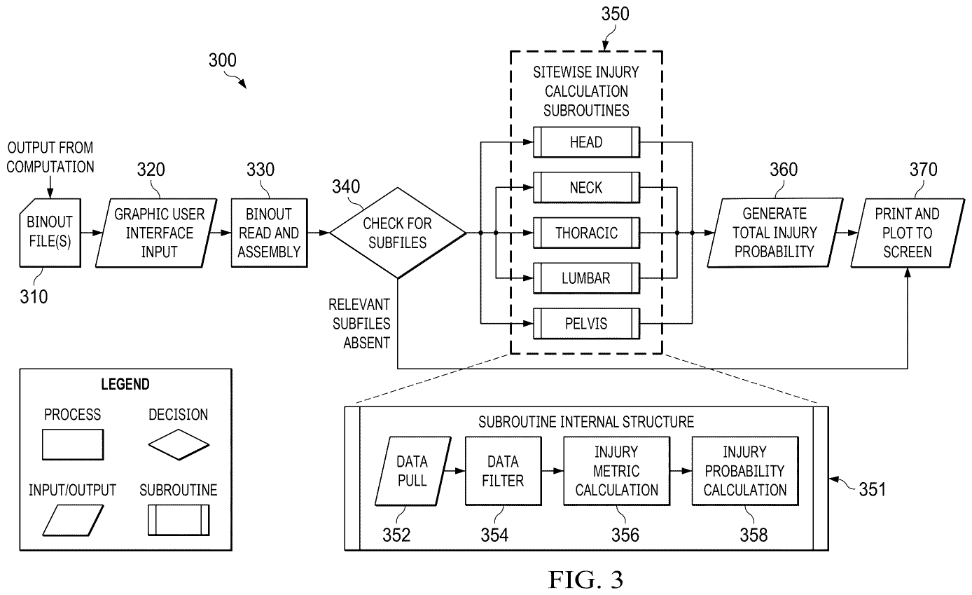

FIG. 2 illustrates a block diagram of an example of a biomechanical injury analyzer constructed according to the principles of the disclosure;

FIG. 3 illustrates a flow diagram of an example operational or work flow for determining a total injury probability of the anatomical complex carried out according to the principles of the disclosure;

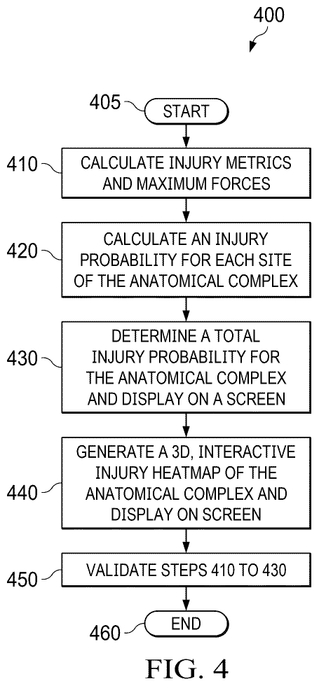

FIG. 4 illustrates a flow diagram of an example method of providing a visual representation of injury probabilities of an anatomical complex carried out according to the principles of the disclosure;

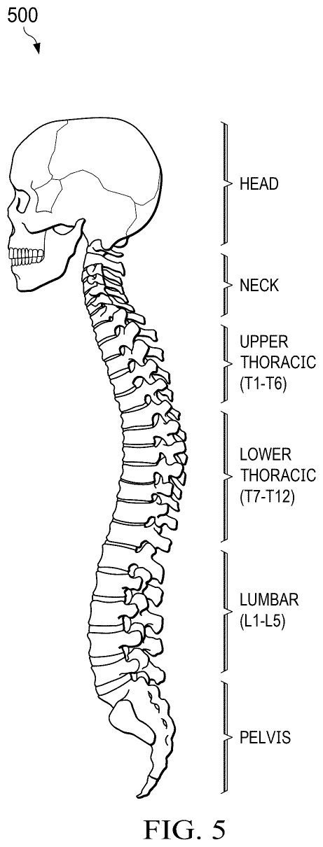

FIG. 5 illustrates an example of the head-neck-spine-pelvis complex; and

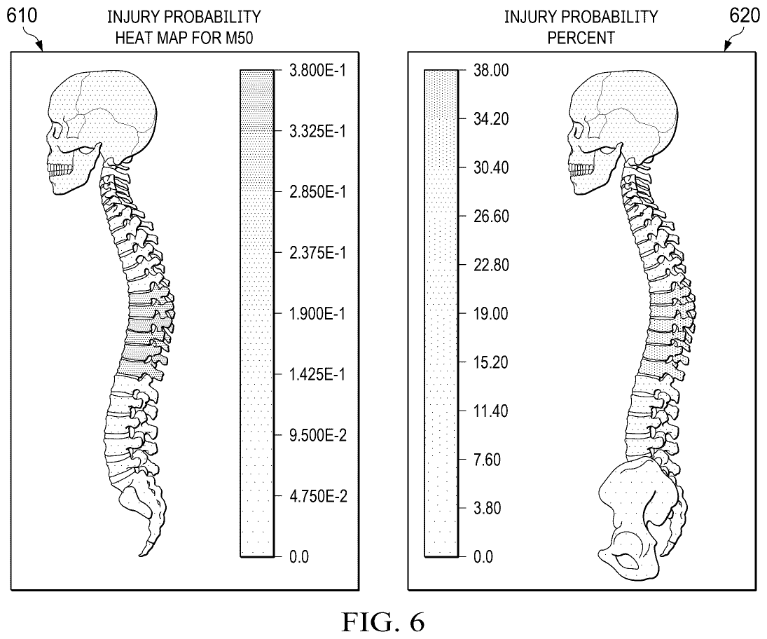

FIG. 6 illustrates an example of both a two dimensional and a three-dimensional heat map.

### DETAILED DESCRIPTION

Simulations to determine how the human body reacts to impact events can be executed on different virtual solvers using an FE-HBM, such as a GHBMC model, to generate simulation data. The simulation data is then further processed to calculate injury probabilities for risk assessment. Calculating the injury probabilities is typically a tedious manual process using specific parameter queries, such as maximum force experienced, rotational moment, etc., that are then compared to relevant injury assessment reference values (IARVs) for each of the particular computations. In addition to being tedious, the injury probability calculations can be inefficiently repetitive and time-consuming when evaluating injury risks from several computations conducted under one multiparametric design study.

The risk assessments are usually shared with stakeholders using the injury metrics and graphs generated based on data from simulators. The benefit of sharing such risk information can be minimal if the stakeholders are unfamiliar with injury metrics or are unable to visualize the predicted injury profile. For example, in the design cycle of an automotive vehicle, decision-makers may range from business leaders and insurance partners to legal advisors and marketing executives. An analyst may find it challenging to inform the site-wise injury risk for an occupant in a proposed design to such a multidisciplinary faculty. Thus, significant gaps in the workflow efficiency persist.

Accordingly, the disclosure provides a biomechanical injury analyzer that automatically analyzes the simulation data generated from simulated impact events to determine risk assessment for an anatomical complex. The simulation data is obtained from a virtual solver that uses human body models (such as FE-HBM) that grant sufficient resolution for analysis. The simulation data is typically binary data provided as a binary output file by the virtual solver. The virtual solver can be a massively parallel processing (MPP) solver. For example, the virtual solver can be a multi-physics simulation tool, such as the LS-DYNA® Multiphysics solver from ANSYS, Inc. of Canonsburg, Pennsylvania. As noted above, the FE-HBM can be a GHBMC model.

The biomechanical injury analyzer provides a comprehensive analysis of a complete anatomical complex and provides an interactive display of a heat map that illustrates injury risks as a macro of the anatomical complex instead of just focusing on a particular body part or site. The displayed heat map provides a communication tool for communicating injury risks over multiple stakeholders. Thus, instead of being tailored to a particular group, the biomechanical injury analyzer provides a universal way to communicate injury risks to diverse parties.

In addition to being an efficient data analysis tool and an effective communication tool, the biomechanical injury analyzer is also a predictive analytic biodynamic modeling tool that allows specific injury risk prediction. The biomechanical injury analyzer, therefore, provides a single solution for risk analysis, prediction, and communication.

As disclosed in further details below, the biomechanical injury analyzer calculates injury metrics and maximum forces for an anatomical complex of the human body, calculates associated injury probabilities, and generates a visual injury heat map of the anatomical complex for display. In addition, the biomechanical injury analyzer determines a total injury probability of the anatomical complex. The biomechanical injury analyzer can automatically perform these operations and in some examples can automatically perform these operations in less than 30 seconds.

FIG. 1 illustrates a block diagram of an example of an injury assessment system 100 constructed according to the principles of the disclosure. The injury assessment system 100 includes a virtual solver 110 and a biomechanical injury analyzer 120. The injury assessment system 100 can be implemented on one or more computing systems or computing devices. The virtual solver 110 can be implemented on one computing system and the biomechanical injury analyzer 120 can be located on a different computing system. The virtual solver 110 and the biomechanical injury analyzer 120 can be communicatively coupled together over a communications network to allow communication of the generated output. The generated output can also be stored in a data storage and retrieved by the biomechanical injury analyzer 120. As disclosed herein, a GUI can be used to identify the desired output, i.e., simulation data, and retrieved for use by the biomechanical injury analyzer 120. The simulation data can also be stored on the biomechanical injury analyzer 120 and retrieved therefrom.

The virtual solver 110 is configured to simulate impact events on a human body and provide simulation data that indicates how the human body reacts to the different impact events. The virtual solver 110 can be a finite element solver. For example, the virtual solver 110 can be a general-purpose multi-physics simulator, such as the above note LS-DYNA® Multiphysics solver. The simulation data can be further processed by a post-processor after being generated by the virtual solver. For example, LS-PrePost can be used to further process the simulation data following the simulation by the LS-DYNA®.

An impact event is a physical event that exerts one or more forces on the human body. The one or more forces can be exerted on a particular portion of the human body or to multiple body portions. The forces and the reaction thereto can be simulated for a particular anatomical complex of the human body and the generated simulation data can correspond to the particular anatomical complex. One such complex is the head-neck-spine-pelvis complex and will be used herein as an example. The spine includes the thoracic (T1-T12) and lumbar (L1-L5) spine. Other examples of anatomical complexes include upper and lower extremities, the rib cage (composed of the twelve pairs of ribs and the sternum), the pelvic skeleton (composed of the ilium, ischium, pubis, sacrum and coccyx), and the pectoral girdle (composed of the clavicles and scapulae).

The virtual solver 110 generates data from simulations of the exertion of the forces on an FE-HBM. A GHBMC model is one representation of a FE-HBM. Simulation data can be generated for various impact conditions. For example, the value of the forces, location of impact, number of impacts, and/or other force parameters can be varied and simulation data can be generated to predict how the human body model may respond for each of the computations using the different force parameters.

The biomechanical injury analyzer 120 is configured to automatically generate a total injury probability of the anatomical complex using the simulation data. The biomechanical injury analyzer 120 also calculates injury probability by local regions or sites, examples including the head, the upper and lower neck, the lumbar spine, etc.). Additionally, the biomechanical injury analyzer 120 is configured to provide an injury heat map for the anatomical complex. Calculating the site injury probabilities and providing an injury heat map can be performed automatically by the biomechanical injury analyzer 120. The heat map can be a three-dimensional (3D), interactive image displayed on a screen. A two-dimensional (2D) heat map image can also be generated and provided for analysis and print. The injury probabilities and heat map (or heat maps) can be generated for each of the different computations to communicate injury risk. FIGS. 2 and 3 provide additional details of a biomechanical injury analyzer.

FIG. 2 illustrates a block diagram of an example of a biomechanical injury analyzer 200 constructed according to the principles of the disclosure. The biomechanical injury analyzer 200 provides an example of the biomechanical injury analyzer 120. The biomechanical injury analyzer 200 is a computing device that includes one or more interfaces represented by interface 210, one or more data storages or memories represented by memory 220, one or more processors represented by processor 230, and a screen 240.

The interface 210 is a communications interface that is configured to send and receive data. Accordingly, the interface 210 includes the necessary circuitry, software, or combination thereof to communicate data. The interface 210 can be a conventional interface of a computing device to send and receive data. For example, the interface 210 can receive simulation data and injury tolerances. A graphical user interface (GUI) 244 displayed on the screen 240 can be used to select the simulation data and an injury tolerance.

The memory 220 is configured to store data including operating instructions that direct the operation of the processor 230. The operating instructions correspond to one or more algorithms directed to calculating injury metrics and maximum forces of an anatomical complex using the simulation data, calculating associated site injury probabilities for each site of the anatomical complex, calculating a total injury probability of the anatomical complex based on the individual injury probabilities of the sites, and providing an injury heat map for the anatomical complex on the display. The total injury probability can also be displayed on the screen.

The processor 230 is configured to perform the operations according to the operating instructions that correspond to the one or more algorithms stored on the memory 220. The algorithms can correspond to the methods/discussion disclosed herein and correspond, for example, to at least a portion of the workflow of FIG. 3 and/or the method of FIG. 4. The operating instructions and displaying of the total injury probabilities can be performed automatically by the processor 230. The processor 230 can be a parallel processor, such as a GPU, or a serial processor, such as a CPU. A combination of different processors can also be used.

The screen 240 is configured to visually display information generated by the processor 230. The information can be, for example, a heat map. The heat map can be a 3D interactive heat map or a 2D heat map. The 2D heat map can be sent to a printer and printed. The screen 240 can be a conventional screen that is used to display data. On the screen 240, the GUI 244 is provided that is configured to receive a selection for the particular simulation data and an injury tolerance. The selection can indicate a file location, a file name, or another type of identifier for the desired simulation data. The screen 240 can be a touch screen that is used for the selection or entering of information to obtain the simulation data and provide the injury tolerance. The screen 240 can also receive inputs via user interface devices, such as a keyboard, a mouse, a mouse pad, a speaker, etc. Received inputs can be used to interact with a displayed heat map.

The simulation data can be stored in the memory 220 or can be stored externally to the biomechanical injury analyzer 200 and retrieved via the interface 110 when selected via the GUI 244. For example, a file location or a file name can be entered via the GUI 244 and retrieved from the memory 220 or from an external location for processing. The file location or file name can also be selected via the GUI 244, such as via a dropdown list. The injury tolerance can also be provided for selection via a dropdown list or can be manually entered by a user. FIG. 3 illustrates a diagram illustrating an example operation of a biomechanical injury analyzer such as biomechanical injury analyzer 200.

FIG. 3 illustrates a flow diagram of an example operational or work flow 300 for determining a total injury probability of the anatomical complex carried out according to the principles of the disclosure. The workflow 300 is executed using a biomechanical injury analyzer, such as biomechanical injury analyzer 120 or 200 of FIGS. 1 and 2. A GUI, such as the GUI 244 of FIG. 2, is used for the work flow 300. The work flow 300 is directed to an anatomical complex that is a head-neck-spine-pelvis complex of the human body and the FE-HBM is a type of GHBMC model. A similar workflow can be used with other anatomical complexes, such as the upper and lower extremities, the rib cage, the pelvic skeleton, and the pectoral girdle. For other anatomical complexes, other algorithms would be used in lieu of those defined in Equations 1 to 9. The algorithms can be developed and validated for use. The algorithms can be published, validated and be well-known algorithms used in the industry. The work flow 300 begins in step 310.

In step 310, binary output files are received by the biomechanical injury analyzer. The binary output files, referred to as binout files, include simulation data corresponding to the reaction of the head-neck-spine-pelvis complex to an impact event. The impact event can be a seat ejection event. A binout file (or a set of binout files generated in one computation) includes simulation data for one impact event. The simulation data can be generated by a virtual solver such as LS-DYNA® and can be processed by a post-processor, such as LS-PrePost, before being received. For example, each binout file can contain the data recorded by the instrumentation of GHBMC models in response to an impact event.

A user, via the GUI, identifies the simulation data, the GHBMC model type, and selects the maximum tolerable probability of injury (i.e, the injury tolerance) for processing in step 320. The user can identify the simulation data by entering or selecting a file path of the binary output files corresponding to the desired simulation data. The GHBMC model type and the injury tolerance can also be entered or selected.

In step 330, the binout files are read and assembled. The virtual solver can be a MPP solver that generates multiple binout files per node and can include multiple subfiles. For example, the binout files can include node-wise kinetic data in a nodal output subfile and element-force data in a section force subfile. The binout files, including the subfiles, are assembled and read using, for example, a computer aided engineering (CAE) library. The CAE library can be the lasso-python open-source library.

A determination is made in step 340 if there are files or subfiles missing for the desired simulation data. In case files or subfiles are missing or data is not available, the user is informed with an error message via the GUI in step 370. Additionally, an error ceases processing of the simulation data file. The biomechanical injury analyzer can then move to the next file in the queue when available and process additional simulation data.

After ensuring the selected simulation data is available, workflow 300 continues to step 350 where the biomechanical injury analyzer evaluates injury by each site of the head-neck-spine-pelvis complex. Each site is evaluated via a subroutine 351. Each time subroutine 351 is executed, data is pulled from appropriate subfiles (i.e., those subfiles for the particular site) in step 352 and filtered in step 354. The data filtering of step 354 can be performed according to standard practices in the industry. For example, the data filtering 354 can be per the SAE J211 recommended practices for channel frequencies (SAE 2022). A Butterworth filter is also an example of a filter that can be used for data filtering. The filtered data is used to calculate the injury criteria in step 356. The injury metrics for each site are used for determining the injury probability of each site in step 358. Each of the particular sites are noted below and an example of calculations for the injury metrics of step 356 and the injury probability of step 358 for each site are provided. Weibull and normal cumulative distribution functions are used in the injury probability calculations. The biomechanical injury analyzer can determine the injury metrics and injury probabilities for the sites using the below corresponding equations. Other methods for determining the injury metrics probabilities, such as other common industry procedures or calculations, could also be used.

Risk of injury is calculated according to injury severity. The Abbreviated Injury Scale (AIS) is an internationally accepted injury severity scoring system. It is used to classify injury by body region on a 6-point scale (AIS1 being a minor injury, AIS6 being maximal). The biomechanical injury analyzer can calculate injury probability for each site of the head-neck-spine-pelvis complex by injury severity score.

### Head

Injury probability of the head is evaluated using the Head Injury Criterion (HIC15). The HIC15 is a weighted exponent of the highest average resultant translational acceleration of the head's center-of-mass in a window of 15 milliseconds or less. It is calculated using Equation (1).

$$HIC 15 = max \{ ( {t}_{2} - {t}_{1} ) \cdot {[ \frac{1}{{t}_{2} - {t}_{1}} {\int }_{{t}_{1}}^{{t}_{2}} a ( t ) d t ]}^{2.5} \} \qquad ( 1 )$$

In Equation 1, t₂−t₁<15 milliseconds and a(t) is the head's acceleration in g's. The biomechanical injury analyzer calculates head injury by first extracting the time history of the resultant acceleration of the head's center-of-gravity. After filtering this acceleration at an SA E channel frequency of 1000 Hz, it is then iteratively integrated across the time history with time limits no greater than 15 milliseconds apart per iteration. The integration can be carried out using the trapezium function in the NumPy Python library. The head's acceleration and the corresponding HIC15 is calculated per iteration.

In step 358, the probability of suffering a head injury is determined using the HIC15. The head injury considered can be a head injury of AIS1 or greater severity. The maximum of all HIC15 calculated can be used to evaluate the heads' injury probability using the injury risk curve as in Equation (2). The determined head injury probability is then printed to screen and written to file in step 370.

$${P}_{h e a d} = 1 - exp ( - {( \frac{HIC 15}{671} )}^{4.34} ) \qquad ( 2 )$$

### Neck

The neck's injury probability is evaluated in step 358 by two sub-segments: the upper neck and lower neck. The atlantoaxial joint's instabilities make up a majority of neck injuries borne in frontal impacts due to the head's kinematics. However, step 358 also considers an additional risk to the cervicothoracic junction, which is susceptible to burst fractures. The biomechanical injury analyzer evaluates the injury probability separately for proximal and distal ends of the neck.

### Upper Neck

Injury probability of C1-C2 is evaluated using the neck injury criterion (Nij). The axial force and frontal moment experienced at C2 are normalized against critical IARVs and summed to calculate the criterion. For a 50th percentile American male, the IARVs are 6806 N for Tension, 6106 N for compression, 310 Nm for flexion and 135 Nm for Extension. Equation (3) provides an example of the calculation.

$${N}_{i j} = \frac{{F}_{Z}}{{F}_{Z i n t}} + \frac{{M}_{Y}}{{M}_{Y i n t}} \qquad ( 3 )$$

Neck injury probability is calculated using the injury risk curve represented by Equation 4, which evaluates injury at an AIS2 or greater severity.

$${P}_{upper - neck} = \frac{1}{1 + exp ( 5.245 - 4.1 \cdot Nij )} \qquad ( 4 )$$

In Equation 4, Nij represents the neck injury criterion. The biomechanical injury analyzer extracts the time history of C2's axial force and neck moment from the section force (secforc) subfile. It then calculates the neck injury criterion as in Equation (3) for every timestep. The maximum of all such calculated criteria is stored as the final Nij. This is then used to calculate the neck injury probability in step 358 using Equation (4). The determined upper neck injury is finally printed to screen and written to file in step 370.

### Lower Neck

The beam criterion is calculated for the lower neck's injury probability. Similar to the neck injury criterion, the beam criterion normalizes the axial force and frontal moment at the cervicothoracic junction against critical force and moment IARVs. The beam criterion is calculated using Equation (5).

$$B C = \frac{{F}_{Z}}{{F}_{Z C}} + \frac{{M}_{Y}}{{M}_{Y C}} \qquad ( 5 )$$

The critical values are 5660 N for tension force, 5430 N for compression force and 141 N-m for moment. The injury probability at a severity of AIS2 or greater for the lower neck is evaluated using the beam criterion with the injury risk function stated in Equation (6).

$${P}_{lower - neck} = \frac{1}{1 + exp ( \frac{1.0261 - B C}{0.2076} )} \qquad ( 6 )$$

The biomechanical injury analyzer extracts the axial force and frontal moment time histories of the T1 joint from the secforc subfile. After filtering the data at a frequency of 600 Hz, the biomechanical injury analyzer then calculates the beam criterion per timestep. Finally, the maximum beam criterion thus calculated is used to evaluate the injury probability using Equation (6). In step 370, the biomechanical injury analyzer prints to screen and writes to file the lower neck injury probability, along with the maximum beam criterion.

### Thoracic

The thoracic vertebrae's injury probability is evaluated by assessing stresses within two thoracic regions. T2-T6 and T7-T12. Two injury risk curves are used for the thoracic vertebrae (one for each region) by impacting each region specimen vertically and registering the force at the distal end (T2 and T7). The injury risk functions thus developed are as in Equations (7a) and (7b).

$${P}_{upper - thoracic} = 1 - exp ( - {( \frac{{F}_{T 2}}{3688} )}^{4.5425} ) \qquad ( 7 a )$$

$${P}_{upper - thoracic} = 1 - exp ( - {( \frac{{F}_{T 7}}{3944} )}^{5.74} ) \qquad ( 7 b )$$

In Equations (7a) and (7b), F is the axial thoracic force in Newtons experienced at T2 and T7, respectively. The biomechanical injury analyzer extracts the time histories of 12 thoracic axial forces from the secforc subfile. The forces are filtered with an SA E channel frequency of 1000 Hz and displayed on the GUI. For calculating injury probabilities, the maximum force experienced at T2 and T7 are computed and then used in Equations (7a) and (7b). Both thoracic region injury probabilities, and the maximum forces of all 12 vertebrae, are printed to screen and written to file in step 370. Thoracic vertebrae fractures are typically deemed to be of AIS3 or greater severity.

### Lumbar

The lumbar vertebrae's injury probability is calculated using axial forces. Two injury risk functions are used to assess vertical impacts on lumbar specimens and the resultant forces at the thoracolumbar and lumbosacral ends are computed. The functions to determine lumbar vertebrae injury are given in Equations (8a) and (8b).

$${P}_{upper - lumbar} = 1 - Φ ( - 3 \text{.298} \cdot log ( \frac{{F}_{L 1}}{5700} ) ) \qquad ( 8 a )$$

$${P}_{upper - lumbar} = 1 - Φ ( - 3 \text{.498} \cdot log ( \frac{{F}_{L 5}}{6800} ) ) \qquad ( 8 b )$$

In Equations (8a) and (8b), F is axial lumbar force in Newtons. The biomechanical injury analyzer extracts the axial force time histories of all five lumbar vertebrae from the secforc subfile. Each is filtered with an SA E channel frequency of 600 Hz. From these, the maximum forces at L1 and L5 are determined and then used to calculate injury probabilities. The maximum of these two probabilities is reported as the injury probability of the entire lumbar segment. All vertebrae's force time histories are displayed and the maximum forces of each along with the injury probabilities are printed on the screen in step 370.

### Pelvis

The pelvis' injury risk function is based on vertical impact testing of whole-body post-mortem human subjects (PMHS). Pelvic fractures are assessed via CT-scans and autopsies. The risk function for pelvic injury probability is stated in Equation (9).

$${P}_{p e l ν i s} = 1 - exp ( - {( \frac{a}{223.39} )}^{2.2371} ) \qquad ( 9 )$$

In Equation 9, ais the acceleration of the sacrum in g's. The biomechanical injury analyzer extracts the resultant acceleration time history of the node representing the pelvic center-of-gravity, which is then filtered at a frequency of 1000 Hz to determine the maximum acceleration to be used in Equation (9). Pelvic injury probability and peak pelvic acceleration are printed on the GUI, and the time history is also displayed on the GUI in step 370.

After determining the injury probability for each site, the biomechanical injury analyzer then calculates a total injury probability for the entire complex in step 360. The total injury probability can be determined according to step 430 of method 400. The biomechanical injury analyzer then displays these injury probabilities to the screen and writes them to a text file in step 370. In addition to the total injury probability, the biomechanical injury analyzer can also generate heat maps corresponding to the injury probabilities for the individual sites. A 3D interactive heat map can be generated and displayed on the screen. FIG. 6 illustrates an example of a 2D heat map 610 and 3D heat map 620 that can be displayed on the screen and can be changed in response to user inputs.

FIG. 4 illustrates a flow diagram of an example method 400 of providing a visual representation of injury probabilities of an anatomical complex carried out according to the principles of the disclosure. Method 400 can be carried out according to operating instructions corresponding to one or more algorithms, including those represented by the Equations disclosed herein. Method 400 can use a biomechanical injury analyzer as disclosed herein to perform one or more of the steps of method 400. Method 400 can be performed automatically by the biomechanical injury analyzer, such as automatically performed by one or more processors of the biomechanical injury analyzer. Method 400 begins in step 405.

In step 410, injury metrics and maximum forces of an anatomical complex are calculated using simulation data. A virtual solver can generate the simulation data. The simulation data consists of the finite element human body model's (FE-HBM) dynamic responses to dynamic impacts.

In step 420, injury probabilities for each site of the anatomical complex is calculated using the corresponding injury metrics, maximum forces, or a combination thereof for each of the sites. The calculations of step 410 and 420 can be performed according to the steps of subroutine 351. Equations 1 to 9 represent examples for calculations performed in steps 410 and 420 for a head-neck-spine-pelvis complex.

In step 430, a total injury probability for the anatomical complex is determined and displayed on a screen of a computing device. The computing device can be part of an injury assessment system, such as system 100. To determine the total injury probability, a composite injury metric for the anatomical complex is used. As an example, the head-neck-spine-pelvis complex 500 is employed as illustrated in FIG. 5. The complex 500 is evaluated as a system made of separate components or sites, connected in series, as shown in FIG. 5. Equating injury to serial system “reliability,” an injury to any of the sites, such as the neck, lower thoracic region, etc., would result in “degradation” of the system. The rationale is that if a subject experienced higher than allowable loads anywhere in the sites of the complex 500, the subject would be deemed injured. Thus, site-wise injury probabilities can be considered analogous to component failure probabilities. The failure probability of the complex 500 can then be calculated using a series reliability equation. The biomechanical injury analyzer calculates and stores the injury probability associated with each of the analyzed injury sites and then calculates the total injury probability as in Equation (10). This total injury probability is printed to screen and also written to file.

TIP_{sys}=1−∪ₚ(1−P(p)),∀pϵsys (10)

In Equation 10, P is the injury probability of individual part p and TIP_{syn} is the Total Injury Probability (TIP) of the analyzed complex. The TIP calculation implicitly assumes that the probability of a site's injury is independent of that of other sites. The pelvic, lumbar and thoracic injury risk curves were developed by extracting the respective structures from cadavers and impact-testing them, isolating each from the rest of the body. However, the head and neck injury risk functions were not developed in such isolation. Given that some overlap between the head and neck's injury assessment is inevitable, the total injury probability metric may overestimate total injury and therefore is treated as a conservative injury metric. The TIP combines the injury probabilities of individual sites of an anatomical complex, such as complex 500, into a single intuitive metric that is broadly applicable to different domains involving dynamic loads.

In step 440, an injury heat map of the anatomical complex is generated and displayed on a screen. The injury heat map is a 3D interactive heat map that can be generated using, for example, the Visual Anatomical Injury Descriptor (Visual Aid) developed by Patrick Gillich.

Thus, in addition to calculating quantitative site-wise injury probabilities, the 3D interactive heat map qualitatively color-plots each site to contrast sites by likelihood of injury. Each site is color-coded, such as on a white-to-red gradient, wherein white represents the lowest local injury probability and red the highest. All sites with a local injury probability higher than the user-defined injury tolerance would also be colored red. Different colors can be used for the color coding instead of the white-to-red gradient. The display of the 3D interactive heat map can be updated or changed in view of one or more user inputs, such as the injury tolerance. Other inputs can include identifying and/or rotating different sites using a user interface device. A GUI provided on the screen can be used to receive the inputs. In addition to the 3D interactive heat map, a two-dimensional heat map can also be created. An example illustration of both a two-dimensional and a three-dimensional heat map are shown in FIG. 6. The heat maps can be saved to a file along with the total injury probability for the anatomical complex.

Validation of the calculation of method 400 is performed in step 450. The accuracy of the biomechanical injury analyzer can be validated by comparing its post-simulation injury probability calculations to the observed rate of injuries in a real-world vertical loading event with known parameters. The real-world data can be computationally simulated for comparison to the calculated results of the biomechanical injury analyzer. For example, a military aircraft seat ejection event loads an occupant vertically (+Gz) with accelerations as high as 25 G and a maximum jolt of 300 G/sec, leading to potential spinal injuries due to axial-compressive trauma. Retrospectively spinal injuries in airmen who initiated and survived aircraft seat ejections were evaluated. These military aircraft ejection seat events are computationally simulated via LS-DYNA using GHB MC models. The biomechanical injury analyzer is then employed to calculate site-wise and total injury probabilities. The results from the simulation study are then compared to the observed percentage of vertebral injuries incurred by aircrew members to validate operation of the biomechanical injury analyzer. A graph can be generated comparing the results and can be saved with the other generated outputs from method 400.

Method 400 then continues to step 460 and ends. Method 400 can be repeated using simulation data corresponding to other force parameters. The simulation data can correspond to one file and method 400 can include queueing and automatically processing multiple files, wherein each of the different files include simulation data corresponding to different simulations using one or more different force parameters.

A portion of the above-described apparatus, systems or methods may be embodied in or performed by various, such as conventional, digital data processors or computers, wherein the computers are programmed or store executable programs of sequences of software instructions to perform one or more of the steps of the methods. The software instructions of such programs or code may represent algorithms and be encoded in machine-executable form on non-transitory digital data storage media, e.g., magnetic or optical disks, random-access memory (RAM), magnetic hard disks, flash memories, and/or read-only memory (ROM), to enable various types of digital data processors or computers to perform one, multiple or all of the steps of one or more of the above-described methods, or functions, systems or apparatuses described herein.

Portions of disclosed embodiments may relate to computer storage products with a non-transitory computer-readable medium that have program code thereon for performing various computer-implemented operations that embody a part of an apparatus, device or carry out the steps of a method set forth herein. Non-transitory used herein refers to all computer-readable media except for transitory, propagating signals. Examples of non-transitory computer-readable media include, but are not limited to: magnetic media such as hard disks, floppy disks, and magnetic tape; optical media such as CD-ROM disks; magneto-optical media such as floptical disks; and hardware devices that are specially configured to store and execute program code, such as ROM and RAM devices. Examples of program code include both machine code, such as produced by a compiler, and files containing higher level code that may be executed by the computer using an interpreter. Configured or configured to means, for example, designed, constructed, and/or programmed, with the necessary logic, algorithms, processing instructions, and/or features for performing a task or tasks.

Those skilled in the art to which this application relates will appreciate that other and further additions, deletions, substitutions and modifications may be made to the described embodiments.

Each of the aspects disclosed in the Summary can include elements of one or more of the below dependent claims in a combination.

What is claimed is:

1. A biomechanical injury analyzer, comprising:
a screen; and
one or more processors to perform operations, the operations including:
calculating injury metrics, maximum forces and maximum moments of an anatomical complex using simulation data, wherein the simulation data is from a simulation of an impact event on a finite element human body model (FE-HBM),
calculating associated site injury probabilities for each site of the anatomical complex using the injury metrics or the maximum forces associated with the site,
calculating a total injury probability of the anatomical complex based on the individual injury probabilities of the sites,
displaying on the screen an injury heat map of the anatomical complex, and
displaying on the screen the total injury probability of the anatomical complex.

2. The biomechanical injury analyzer as recited in claim 1, wherein the FE-HBM is a Global Human Body Model Consortium (GHBMC) model.

3. The biomechanical injury analyzer as recited in claim 1, wherein the simulation is executed on a finite element solver and the simulation data is a binary output file.

4. The biomechanical injury analyzer as recited in claim 1, wherein the simulation data corresponds to one file and the operations further include queueing and automatically processing multiple files, wherein each of the different files include simulation data corresponding to different simulations using one or more different force parameters.

5. The biomechanical injury analyzer as recited in claim 1, further comprising executing a simulation based on actual data and validating both the site injury probabilities and the total injury probability by comparing the site injury probabilities and the total injury probability from the simulation data to site injury probabilities and total injury probability from the actual data.

6. The biomechanical injury analyzer as recited in claim 1, wherein the anatomical complex is a head-neck-spine-pelvis complex.

7. The biomechanical injury analyzer as recited in claim 1, wherein the operations further include receiving an injury tolerance and the heat map is displayed according to the injury tolerance.

8. The biomechanical injury analyzer as recited in claim 7, wherein the operations further include providing a GUI on the screen and receiving an input via the GUI for selecting the simulation data and an input for the injury tolerance.

9. The biomechanical injury analyzer as recited in claim 1, wherein the injury heat map is interactive and qualitatively colors each site of the anatomical complex according to the corresponding site injury probability.

10. The biomechanical injury analyzer as recited in claim 1, wherein the operations further include providing the total injury probability of the anatomical complex for display on the screen.

11. An injury assessment system, comprising:
a virtual solver configured to simulate impact events on a human body and provide simulation data that indicates how the human body reacts to the impact events; and
a biomechanical injury analyzer configured to automatically generate a total injury probability of the anatomical complex using the simulation data.

12. The system as recited in claim 11, wherein the biomechanical injury analyzer includes one or more processors to perform operations, the operations including automatically generating the total injury probability by:
calculating injury metrics and maximum forces of an anatomical complex using the simulation data,
calculating injury probabilities for each site of the anatomical complex based on the injury metrics and the maximum forces, and
calculating the total injury probability of the anatomical complex using each of the individual injury probabilities of the sites.

13. The system as recited in claim 12, wherein the operations further include:
generating a three-dimensional, interactive injury heat map of the anatomical complex, and
displaying the total injury probability and the injury heat map on a screen of the system.

14. The system as recited in claim 13, wherein the operations further include receiving an injury tolerance and displaying the injury heat map is according to the injury tolerance.

15. The system as recited in claim 12, wherein the simulation data corresponds to one file and the operations further include queueing and automatically processing multiple files, wherein each of the different files include simulation data corresponding to different simulations using one or more different force parameters.

16. The system as recited in claim 12, wherein the operations further include executing a simulation based on actual data and validating both the site injury probabilities and the total injury probability by comparing the site injury probabilities and the total injury probability from the simulation data to site injury probabilities and total injury probability from the actual data.

17. A method of providing a visual representation of injury probabilities of an anatomical complex, comprising:
calculating injury metrics and maximum forces of an anatomical complex using simulation data, wherein the simulation data is from a simulation of an impact event on a finite element human body model (FE-HBM);
calculating injury probabilities for each site of the anatomical complex based on the injury metrics and maximum forces generating a three-dimensional, interactive heat map using the injury probabilities for each of the sites; and
displaying the injury heat map of the anatomical complex of a screen of a computing device.

18. The method as recited in claim 17, further comprising updating the display of the injury heat map in response to user inputs received via a graphical user interface (GUI) on the screen.

19. The method as recited in claim 18, wherein the user input is an injury tolerance received via the GUI.

20. The method as recited in claim 17, further comprising calculating a total injury probability of the anatomical complex based on the individual injury probabilities of the sites and displaying the total injury probability on the screen.
